(19) United States
(12) Patent Application Publication
(10) Pub. No.: US 2005/0277978 A1
(43) Pub. Date: Dec. 15, 2005

(54) Three-dimensional coils for treatment of vascular aneurysms

(75) Inventor: E. Skott Greenhalgh, Wyndmoor, PA (US)

Correspondence Address: SYNNESTVEDT & LECHNER, LLP, 2600 ARAMARK TOWER, 1101 MARKET STREET, PHILADELPHIA, PA, 191072950, US

(73) Assignee: Secant Medical, LLC, Perkasie, PA (US)

(21) Appl. No.: 11/148,601

(22) Filed: Jun. 9, 2005

Related U.S. Application Data
(60) Provisional application No. 60/578,424, filed on Jun. 9, 2004.

(51) Int. Cl. A61M 29/00

(52) U.S. Cl. 606/200

(57) ABSTRACT

A coil for the treatment of vascular aneurysms is disclosed. The coil is formed from filamentary members interlaced to form a three-dimensional substrate. The substrate is deformable between a collapsed state wherein it fits within a catheter, and an expanded state, which it assumes once deployed from the catheter into the vascular aneurysm. The three-dimensional shapes of the substrate include flat sheets biased into a helix or a tubular forms biased into figure eight loops, helical loops or a sinusoid. Bioactive characteristics may be imparted to the coil through the use of coatings or materials that provoke a healing response in living tissue.

### FIELD OF THE INVENTION

This invention relates to an intravascular device used in the treatment of aneurysms, and especially in the occlusion of cerebrovascular saccular aneurysms.

### BACKGROUND OF THE INVENTION

Saccular aneurysms occur in arteries in the body and comprise a sack-like formation of the artery wall which extends outwardly from, for example, a bifurcation point between the arterial branches. The aneurysm has a neck forming the juncture with the artery and is capped by a dome. During formation of the aneurysm, the arterial internal elastic lamina disappears at the base of the neck, the sack wall thins and weakens and connective tissue replaces smooth-muscle cells. The aneurysm tends to rupture at the dome and bleeding ensues.

Rupture of a cerebrovascular saccular aneurysm is especially serious due to the associated high mortality rate (10% within the first day of rupture, 25% within three months) and the major neurological deficits experienced by those who survive the initial hemorrhage. Naturally, therapeutic treatment of cerebrovascular aneurysms emphasizes preventing the initial rupture.

Intravascular Catheter Treatment Technique

Intravascular catheter techniques for treating saccular aneurysms are discussed in U.S. Pat. No. 5,122,136, hereby incorporated by reference, and U.S. Pat. No. 6,010,498, also hereby incorporated by reference.

The techniques described in these patents can be summarized with reference to FIGS. 1 and 2, which show a saccular aneurysm 20 formed in an artery 22 at a bifurcation point 24. The treatment techniques involve positioning a catheter 26 at the artery bifurcation point 24, the catheter tip 28 extending partially into the neck 30 of the aneurysm 20. Once the catheter is in position, a length of platinum or platinum alloy wire 32 is snaked through the catheter's lumen 34 through the aneurysm neck 30 and into the aneurysm 20. The wire 32 has a length between 0.4 and 20 inches (1 and 50 cm), is relatively thin (between 0.001-0.005 inches in diameter) and flexible and loops and tangles randomly as it is packed into the aneurysm. Blood which would normally circulate under pressure into the aneurysm, causing it to enlarge, weaken and rupture, begins to form clots 36 on the platinum wire tangle and eventually the clots merge and enlarge to form an occlusion 38 (see FIG. 2) which seals off the aneurysm from the blood flow, preventing further enlargement and rupture.

Once the appropriate length of wire is positioned in the aneurysm and the occlusion has been formed, the wire 32 is released at or near the neck 30 of the aneurysm and the catheter is withdrawn (FIG. 2). Wire release is effected by any one of several means, for example, mechanical means or electrolytic means.

While this catheter technique holds great promise of effective treatment for preventing aneurysm rupture, especially cerebrovascular saccular aneurysms, it may be significantly improved by the use of three-dimensional coils that may also have bioactivity characteristics as described herein below.

### SUMMARY OF THE INVENTION

The invention concerns a coil for treatment of vascular aneurysms, the coil being deliverable into an aneurysm of a vascular vessel through a catheter. The coil comprises a plurality of flexible, resilient filamentary members interlaced with one another to form an elongated substrate. The filamentary members bias the substrate into a three-dimensional expanded state. The interlaced filamentary members define a plurality of interstices dispersed on the substrate providing sites for coagulation of blood when the substrate is in the expanded state. The substrate is expandable from a collapsed state (wherein the substrate fits within the catheter), to the expanded state wherein the substrate expands to substantially fill the aneurysm, the substrate assuming the expanded state upon release from the catheter into the aneurysm.

In one embodiment, the filamentary members are interlaced to form a substantially flat sheet biased into a helically shaped tube when in the expanded state. In another embodiment, the substrate comprises a tube, the filamentary members being interlaced preferably by braiding to form the tube. The tube may be biased into any number of various shapes including a helix, a figure eight and a sinusoid.

For both embodiments, the filamentary members may include first filamentary members that have a high elastic modulus and second filamentary members comprised of a material that provokes a strong healing response in living tissue. The high elastic modulus of the first filamentary members provides resiliency for expansion of the substrate to the expanded state, and the healing response provoked by the second filamentary members increases the bioactivity of the coil by promoting blood coagulation on the substrate as well as inter-growth of living tissue with the substrate.

Alternately, the bioactive characteristics of the coil may be increased by coating the filamentary members with a compound, such as thrombin or collagen, that provokes a healing response in living tissue.

In another embodiment, the substrate is substantially covered by a porous membrane attached to the filamentary members. The membrane is formed of non-woven fibrous tendrils, the tendrils being in overlapping and tangled relation to form additional interstices providing further sites for coagulation of blood thereon.

### BRIEF DESCRIPTION OF THE DRAWINGS

FIGS. 1 and 2 are sectional views of a vascular aneurysm treated by a device according to the prior art;

FIGS. 3A-3C are perspective views of various embodiments of three-dimensional coils according to the invention for treatment of vascular aneurysms;

FIGS. 4 and 5 are sectional views of a vascular aneurysm being treated using a three-dimensional coil according to the invention;

FIG. 6 is a perspective view of another embodiment of a three-dimensional coil according to the invention;

FIG. 7 is a perspective view of the coil shown in FIG. 6 being deployed from a catheter; and

FIGS. 8 and 9 are perspective views of other embodiments of three-dimensional coils having membrane coverings according to the invention.

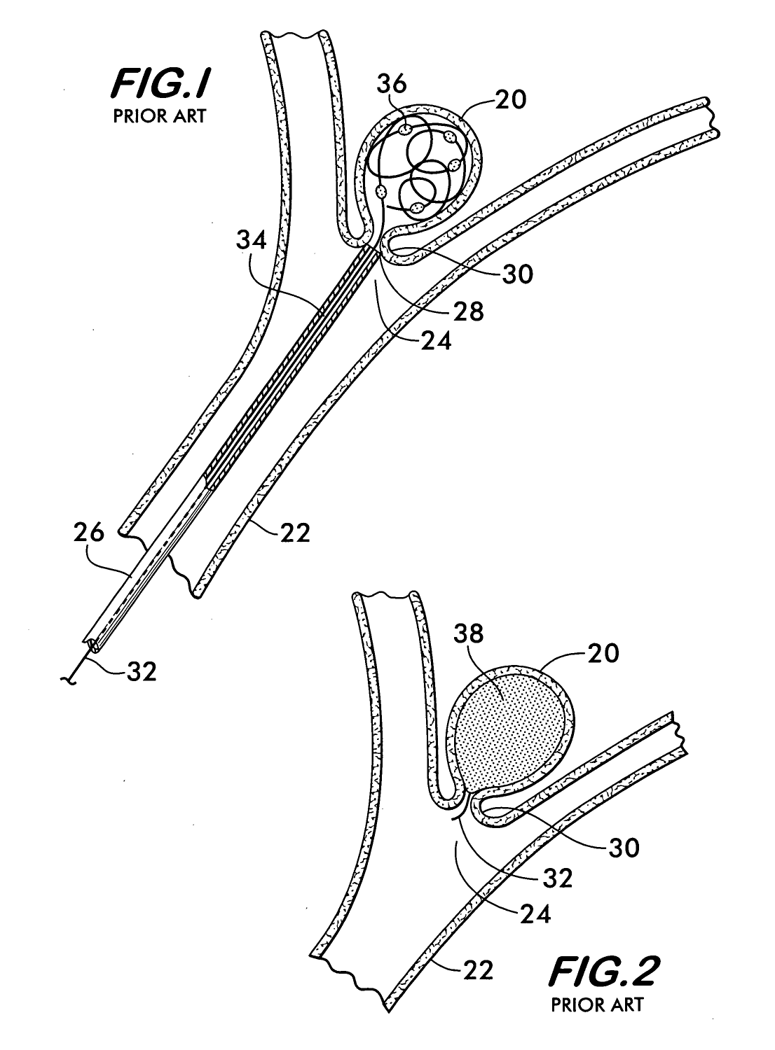

### DETAILED DESCRIPTION OF THE EMBODIMENTS

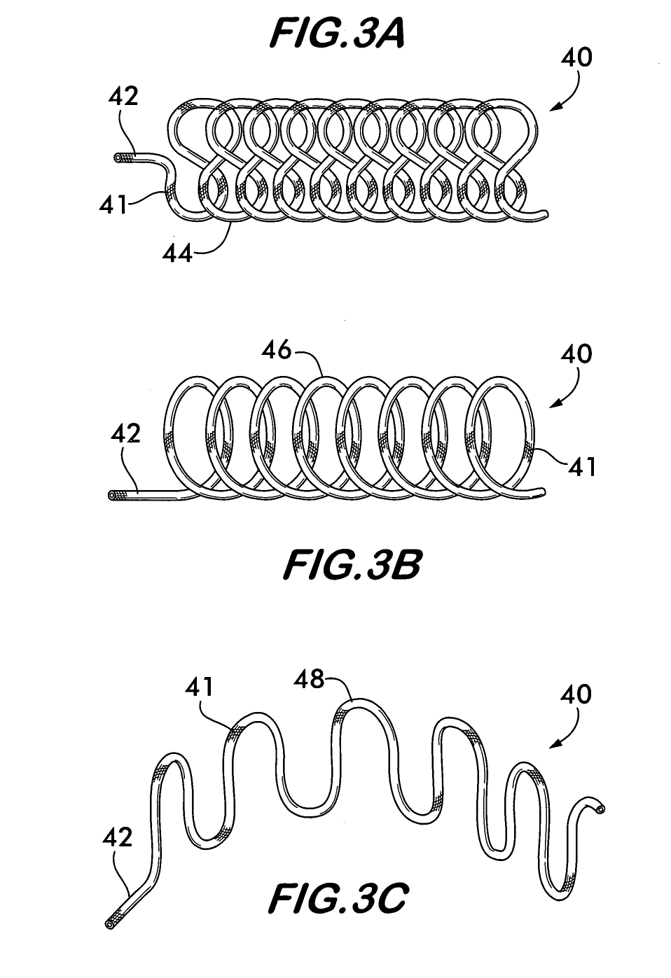

FIGS. 3A-3C show various embodiments of a three-dimensional coil 40 according to the invention used for the treatment of vascular aneurysms. Coil 40 is formed by interlacing flexible, resilient filamentary members 41 to form a tubular substrate 42, the filamentary members being biased so that the substrate 42 nominally assumes a three-dimensional expanded state, in this example, a tubular shape, when unconstrained. Interlacing of the filamentary members for the tubular substrate 42 is preferably by braiding, but weaving and knitting are also feasible. Braiding provides great flexibility, allowing the coil to be easily manipulated into a collapsed state so that it may fit within the lumen of a catheter for delivery to a vascular aneurysm, yet readily expand into its nominal three-dimensional state when released from the catheter as described below.

The tubular substrate itself may also be biased into a number of different shapes. The shapes provide for more orderly deployment of the coil within an aneurysm. In the examples shown, FIG. 3A illustrates a coil biased into a continuous series of loops describing a plurality of “figure eights” 44. FIG. 3B illustrates a helically shaped coil 46, and a sinusoidal shaped coil 48 is shown in FIG. 3C.

The filamentary members 41 interlaced to form the tubular substrate 42 are preferably platinum wires having diameters less than 0.001 inches. Other bio-compatible metals are also feasible including stainless steel, tantalum, elgiloy and nitinol. Metal filaments are advantageous because they have a high elastic modulus which provides resiliency for expansion of the substrate into the expanded state. The metals also have high yield strengths and are therefore readily biasable into the complex curves required by the aforementioned three-dimensional shapes which the coil 40 assumes. Biasing of metal filaments is readily accomplished by annealing or by cold working so that they take a permanent set.

Tubular substrate 42 may also be formed from interlaced polymer filaments such as polytetrafluoroethylene, nylon, polyester, polypropylene or other bio-compatible synthetics. These materials increase the bioactive characteristics of the coil (described below) and are also readily biasable into a desired expanded shape so as to form the three-dimensional structures illustrated in FIGS. 3A-3C. Biasing may be by cold working, chemical treatment or by heat setting to achieve the desired shape. Bio-absorbable materials such as polyglycolic acid and polylactic acid, may also be used.

Furthermore, both metal and non-metal filaments may be interlaced together to form the three-dimensional coil 40. Such a combination allows the advantages of both types of filaments to be realized, as described further below in specific examples.

Additionally, three-dimensional coils 40 may have a skeletal structure positioned within the tubular substrate 42. The structure may be formed of any of various materials appropriate to the function of the structure. For example, metal wire in the form of a helix may be inserted to increase the biasing force and ensure that the coil 40 expands upon release from constraints. Similarly, high bulk filaments of polyester, nylon or collagen may be added to increase clotting and healing functions.

Braided three-dimensional coils 40 display the following advantages over simple coils that are merely single strand wires formed into a particular shape.

The braided three-dimensional coils have great flexibility, allowing them to conform readily to any shape and thereby substantially fill an aneurysm.

The braided tubular substrate 42 that forms the basis of each three-dimensional coil 40 has much greater girth than a simple wire or filament, thus, a shorter length of a three-dimensional coil will occupy more space within an aneurysm than the simple wire.

The girth of the coil tends to make the portions of the coil interfere and tangle with one another, thereby helping keep the coil within the aneurysm.

The large girth coupled with the propensity of coil portions to tangle and interfere allows the three-dimensional coil to be used even with aneurysms having relatively large diameter necks without fear that the coils will become dislodged and extend from the aneurysm.

The flexibility of the three-dimensional coil allows it to collapse and accommodate shrinkage of the aneurysm space which occurs as the aneurysm heals.

The three-dimensional coils have great surface area and form a lattice of interstitial openings that promote blood clotting and healing of the aneurysm.

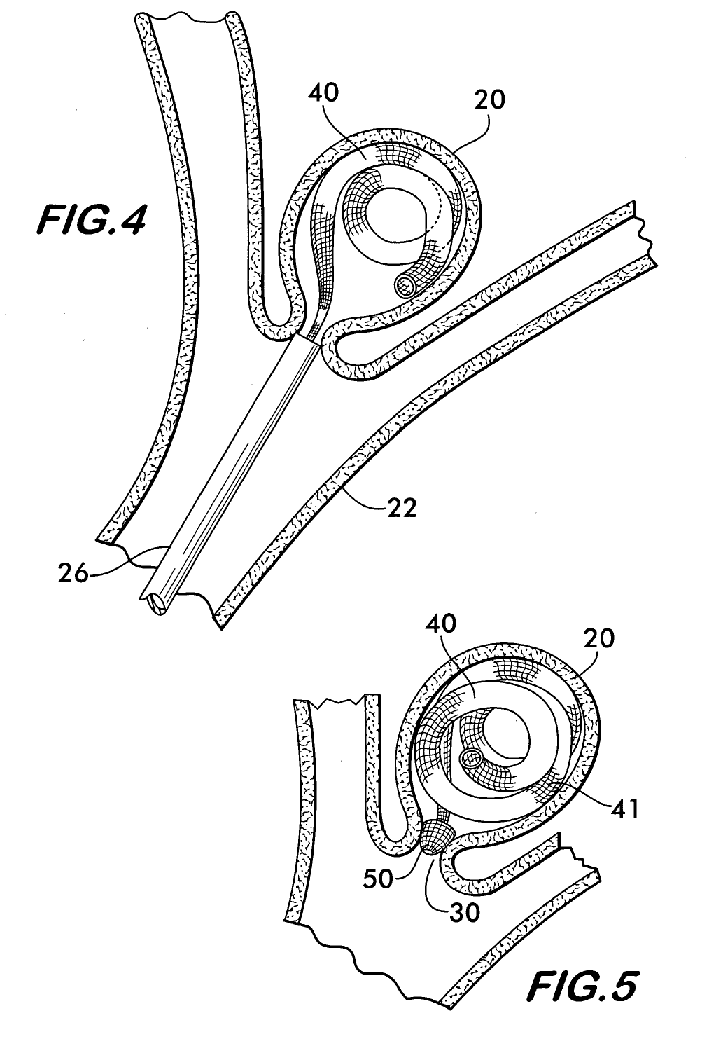

The three-dimensional coils 40 expand radially by factors as high as five when released from constraints, for example, when delivered to an aneurysm 20 from a catheter 26 as shown in FIG. 4. This large expansion ratio, coupled with the biased shape of the braided tubular substrate 42 leads to the capability of delivering a relatively large volume of wire into an aneurysm through a relatively small diameter catheter.

As shown in FIG. 5, an anchor 50 may be attached to the coil 40. The anchor 50 is preferably attached to an end of the coil so that it lodges in the neck 30 of the aneurysm 20. Anchor 50 is preferably formed of interlaced filaments of bio-compatible material such as polypropylene that have an affinity for living tissue. Such materials naturally provoke a healing reaction and will adhere to the aneurysm wall and prevent the end of the coil 40 from dislodging itself from the aneurysm. Alternately, the anchor 50 may be formed by a bioactivity coating, such as collagen or thrombin, applied to the filamentary members 41 forming the coil 40. Such coatings enhance the affinity of the filaments for living tissue and provoke a healing response to secure the coil within the aneurysm.

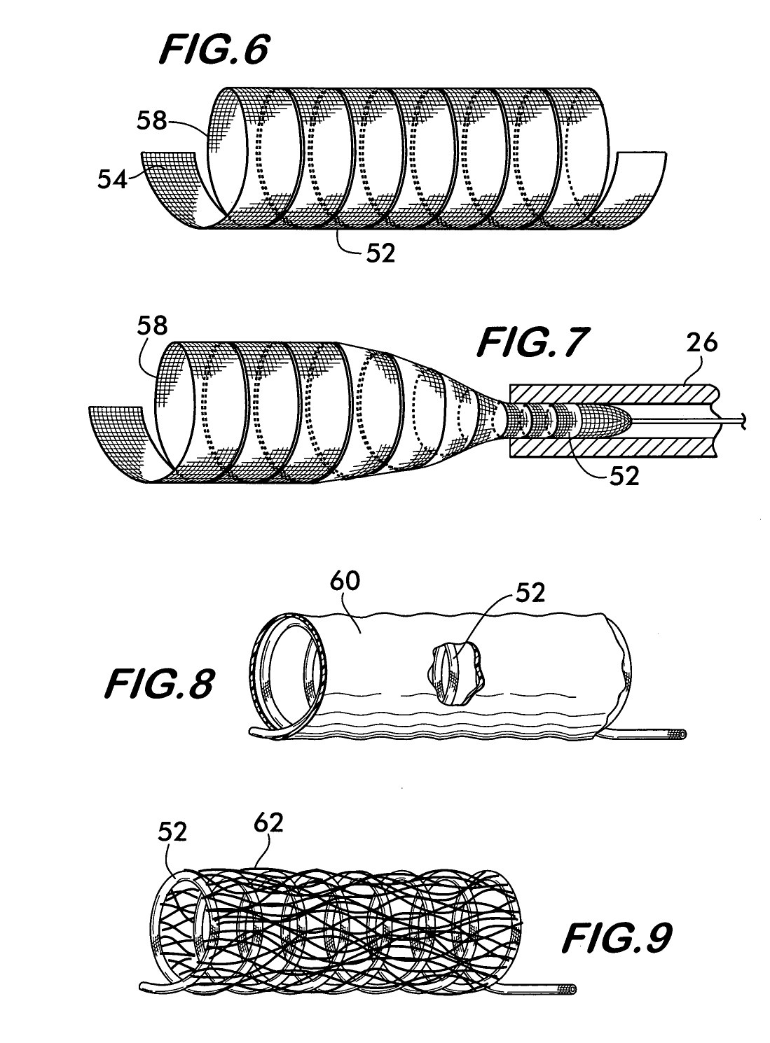

FIG. 6 shows another embodiment of a three-dimensional coil 52 according to the invention. Three-dimensional coil 52 is formed by interlacing filamentary members 54 into a substantially flat sheet that is biased into a helically shaped tubular substrate 58 when in its expanded state. Preferably, the filaments are interlaced by weaving, but braiding and knitting are also feasible. The filaments may be small diameter metal wires comprised of bio-compatible metals such as platinum, tantalum, stainless steel, nitinol and elgiloy. Polymer filaments such as polytetrafluoroethylene, polypropylene, polyethylene, polyester and nylon are also feasible as are the various bio-absorbable polymers. The biasing means which forms the sheet into the helical tubular shape will be appropriate to the material, with cold working or heat annealing being preferred for the metals and chemical and heat setting being feasible for the polymers.

FIG. 7 shows the three-dimensional coil 52 being deployed from a catheter 26. As it leaves the constraints of the catheter, the coil 52 forms itself into the expanded state of the helical coil 58, increasing in volume between 2 and 5 times over its collapsed configuration within the catheter.

The helical tubular substrate 58 enjoys the same advantages as enumerated above for the braided tubular substrate 42. Both embodiments 40 and 52 of the three-dimensional coil according to the invention may be formed by a combination of filamentary members that vary in material properties and physical properties. For example, polymer filaments may be interbraided or interwoven with metal filaments, and filaments, either metal or polymer, having different diameters may be used to provide particular properties for a coil, such as filament density, radiopacity, coil stiffness, increased biasing force and the like.

Any of the three-dimensional coil embodiments described above may be “bioactivity” coils configured to have increased bioactivity to promote more aggressive tissue affinity or blood clotting response and thereby faster aneurysm healing and shrinkage. Increased bioactivity is achieved by coating the coils or the filaments comprising the coils with polymer substances, such as polypropylene, polylactic acid, polyglycolic acid, polyurethane, collagen, thrombin and the like that are known to provoke aggressive healing reaction of living tissue.

As shown in FIG. 8, the coatings may be applied as a membrane 60 adhered to the coil or as a coating on the filamentary members comprising the coil.

FIG. 9 shows a preferred membrane 62 as disclosed in published U.S. Application No. US-2004-0051291-A1, US-2003-0195611-A1, and US-2003-0211135-A1, all of which are hereby incorporated by reference. Membrane 62 is commercially known as “Nanoskin Polymer Membrane” and produces a covering of non-woven fibrous tendrils less than 100 micrometers in diameter. The random overlapping and tangling of the tendrils forms millions of interstices that provide additional sites for blood coagulation, can accept compounds for therapeutic delivery or for increasing bioactivity (collagen, thrombin and the like). The tendrils are formed of various polymers such as polyurethane, polyester, nylon, polypropylene, polyethylene and silicone as well as bioabsorbable materials such as polyglycolic acid, polylactic acid, PLLA, PCL, PDI and PDS.

The bioactivity three-dimensional coil enjoys all of the advantages of the coils described above and adds the ability to accelerate the healing process.

Bioactivity of a particular coil may also be increased as mentioned above, by including in the coil filaments of material that are known to provoke a healing response. For example, polypropylene filaments may be braided with stainless steel filaments to form a three-dimensional braided coil as described above. The steel filaments, due to their high elastic modulus, provide relatively large biasing forces to expand the coil into its desired shape while the polypropylene filaments provide increased bioactivity, polypropylene being known as a material that provokes a particularly strong healing response from living tissue.

Other bioactive coatings for the three-dimensional coils according to the invention are also feasible. For example, radioactive agents, which stimulate cell growth by their particle emissions, may be employed, as well as a hydrogel coating which swells when exposed to blood and cause the coils to adhere to one another.

Three-dimensional coils and such coils that display increased bioactivity due to coatings or material choice provide significant advantage in the treatment of vascular aneurysms because they fill the volume of the aneurysm and provide great surface area for coagulation and intergrowth of living tissue to promote healing and elimination of the aneurysm as a life threatening disorder.

1. A coil for treatment of vascular aneurysms, said coil being deliverable into an aneurysm of a vascular vessel through a catheter, said coil comprising a plurality of flexible, resilient filamentary members interlaced with one another to form an elongated substrate, said filamentary members biasing said substrate into a three-dimensional expanded state, said interlaced filamentary members defining a plurality of interstices dispersed on said substrate providing sites for coagulation of blood thereon when said substrate is in the expanded state, said substrate being expandable from a collapsed state wherein said substrate fits within the catheter, to said expanded state wherein said substrate expands to substantially fill the aneurysm, said substrate assuming said expanded state upon release from said catheter into said aneurysm.

2. A coil according to claim 1, wherein said filamentary members are interlaced to form a substantially flat sheet biased into a helically shaped tube when in said expanded state.

3. A coil according to claim 2, wherein said filamentary members are interlaced by a technique selected from the group consisting of weaving, knitting and braiding.

4. A coil according to claim 3, wherein said filamentary members comprise first and second of said filamentary members, said first filamentary members having a high elastic modulus thereby providing resiliency for expansion of said substrate to said expanded state, said second filamentary members comprising a material that provokes a strong healing response in living tissue for promoting blood coagulation on said substrate.

5. A coil according to claim 3, wherein said filamentary members are coated with a compound that provokes a healing response in living tissue.

6. A coil according to claim 5, wherein said compound is selected from the group consisting of polypropylene, polylactic acid, polyglycolic acid, polyurethane, hydrogel, radioactive agents, thrombin and collagen.

7. A coil according to claim 1, wherein said substrate is substantially covered by a porous membrane attached to said filamentary members and being formed of non-woven fibrous tendrils, said tendrils being in overlapping and tangled relation to form additional interstices providing further sites for coagulation of blood thereon.

8. A coil according to claim 1, further comprising an anchor attached to said substrate, said anchor being formed of a material that provokes a healing response in living tissue, said anchor for attaching said substrate to said vascular vessel within said aneurysm.

9. A coil according to claim 8, wherein said anchor is attached to an end of said substrate.

10. A coil according to claim 8, wherein said anchor is formed from interlaced filamentary members.

11. A coil according to claim 1, wherein said substrate comprises a tube, said filamentary members being interlaced by braiding to form said tube.

12. A coil according to claim 11, wherein said braided tube comprises first and second of said filamentary members, said first filamentary members having a higher elastic modulus than said second filamentary members thereby providing resiliency for expansion of said substrate, said second filamentary members comprising a material that provokes a stronger healing response in living tissue than said first filamentary members for promoting blood coagulation on said substrate.

13. A coil according to claim 11, further comprising a coating for said filamentary members of a compound that provokes a healing response in living tissue.

14. A coil according to claim 11, further comprising an anchor attached to an end of said tube, said anchor being formed of a material that provokes a healing response in living tissue, said anchor attaching said end of said tube to said vascular vessel within said aneurysm.

15. A coil according to claim 14, wherein said anchor is formed from interlaced filamentary members.

16. A coil according to claim 11, wherein said tube is biased into a shape selected from the group consisting of a helix, a figure eight and a sinusoid.

17. A coil according to claim 11, wherein said tube is substantially covered by a porous membrane attached to said filamentary members and being formed of non-woven fibrous tendrils, said tendrils being in overlapping and tangled relation to form additional interstices providing further sites for coagulation of blood thereon.

18. A coil according to claim 1, wherein said substrate comprises a tube, said filamentary members being interlaced by knitting to form said tube.

19. A coil for treatment of vascular aneurysms, said coil being deliverable into an aneurysm of a vascular vessel through a catheter, said coil comprising a plurality of flexible, resilient filamentary members interwoven with one another to form an elongated, substantially flat substrate, said substrate being biased into a three-dimensional helical shape, said interwoven filamentary members defining a plurality of interstices on said substrate providing sites for coagulation of blood, said substrate being expandable from a collapsed state wherein said substrate interfits within said catheter, to an expanded state wherein said substrate expands to substantially fill said aneurysm, said substrate expanding upon release from said catheter into said aneurysm, an anchor being attached to said substrate, said anchor being formed of a material that provokes a healing response in living tissue, said anchor being attachable to said vascular vessel within said aneurysm.

20. A coil according to claim 19, wherein said substrate is substantially covered by a porous membrane attached to said filamentary members and being formed of non-woven fibrous tendrils, said tendrils being in overlapping and tangled relation to form additional interstices providing further sites for coagulation of blood thereon.

21. A coil for treatment of vascular aneurysms, said coil being deliverable into an aneurysm of a vascular vessel through a catheter, said coil comprising a plurality of flexible, resilient filamentary members interbraided with one another to form an elongated, tubular substrate, said substrate being biased into a three-dimensional shape, said interbraided filamentary members defining a plurality of interstices on said substrate providing sites for coagulation of blood thereon, said substrate being expandable from a collapsed state wherein said substrate interfits within said catheter, to an expanded state wherein said substrate expands to substantially fill said aneurysm, said substrate expanding upon release from said catheter into said aneurysm, an anchor being attached to an end of said substrate, said anchor being formed of a material that provokes a healing response in living tissue, said anchor serving to attach said end of said substrate to said vascular vessel within said aneurysm.

22. A coil according to claim 21, wherein said tubular substrate is biased into a shape selected from the group consisting of a helix, a figure eight and a sinusoid.

23. A coil according to claim 21, wherein said tubular substrate is substantially covered by a porous membrane formed of non-woven fibrous tendrils attached to said filamentary members, said tendrils being in overlapping and tangled relation to form additional interstices providing further sites for coagulation of blood thereon.
